(19) United States
(12) Patent Application Publication
(10) Pub. No.: US 2026/0207356 A1
(43) Pub. Date: Jul. 23, 2026

(54) SHUTTLE LOCK

(71) Applicant: Bulldog Tools, Inc., Lewisburg, OH (US)

(72) Inventor: Robert Meyer, Lewisburg, OH (US)

(21) Appl. No.: 19/456,863

(22) Filed: Jan. 22, 2026

Related U.S. Application Data
(60) Provisional application No. 63/748,103, filed on Jan. 22, 2025.

(51) Int. Cl. A61F 2/78

(52) U.S. Cl. CPC A61F 2/78

(57) ABSTRACT

A shuttle lock for use with an artificial limb comprises an upper assembly, a lower assembly, and a locking mechanism. The upper assembly includes a cutout for a channel with a wide portion and a narrow portion, and a lip around an outer edge of the upper assembly such that a groove does not extend to the outer edge of the upper assembly. The lower assembly includes a platform that surrounds a portion of an aperture, two spring pockets, a cutout complementary to the cutout of the upper assembly. A locking-mechanism pocket extends from the spring pockets to surround the aperture and includes a middle portion and two side portions that flank the middle portion. The middle portion is raised above the side portions and includes a width less than a width of the platform. A bottom of the lower assembly includes a sunken portion terminating before coupling holes.

### CROSS REFERENCE TO RELATED APPLICATIONS

This application claims the benefit of U.S. Provisional Patent Application Ser. No. 63/748,103, filed Jan. 22, 2025, having the title TORSION ADAPTER in the Application Data Sheet, the entire disclosure of which is hereby incorporated herein by reference.

### BACKGROUND

Various aspects of the present disclosure relate generally to prosthetic implants and more specifically to a shuttle lock for a prosthetic implant.

A prosthetic implant is an artificial device that replaces a missing body part, including limbs. Some limb prosthetic implants couple via a shuttle lock that allows for release of the limb prosthetic implant from a coupling system.

### BRIEF SUMMARY

According to aspects of the present disclosure, shuttle lock for use with an artificial limb comprises an upper assembly, a lower assembly, and a locking mechanism. The upper assembly includes an aperture for receiving a coupler; a cutout for a channel, wherein the cutout includes a wide portion and a narrow portion; a groove on a surface of the upper assembly, wherein the groove extends radially from the aperture toward an outer edge of the upper assembly; and a lip around the outer edge of the upper assembly such that the groove does not extend to the outer edge of the upper assembly. The lower assembly includes an aperture that aligns with the aperture of the upper assembly when the upper assembly and the lower assembly are coupled together; a platform that surrounds a portion of the aperture of the lower assembly, wherein the platform includes a width; a first spring pocket located co-radially with the aperture of the lower assembly; and a second spring pocket located non-radially with the aperture of the lower assembly, wherein the second spring pocket is longer than the first spring pocket. The lower assembly further includes a cutout, wherein the cutout includes a wide portion and a narrow portion, such that when the upper assembly and the lower assembly are coupled together, the channel is formed. A locking-mechanism pocket extends from the first spring pocket and the second spring pocket and surrounds the aperture of the lower assembly, includes a middle portion, and includes two side portions that flank the middle portion, wherein the middle portion is raised compared to the side portions and includes a width less than a width of the platform. A bottom of the lower assembly includes a collar surrounding the aperture of the lower assembly, wherein the collar protrudes away from a surface of the bottom, includes coupling holes that are flush with the surface of the bottom, and includes a sunken portion extending from the collar and terminates before the coupling holes. The locking mechanism includes a lock plate with an aperture that fits around the platform. The locking mechanism further includes a pin that fits into the channel and abuts the lock plate such that when a user activates the pin, the lock plate traverses within the locking-mechanism pocket to unlock the shuttle lock. A first spring within the first spring pocket biases the lock plate linearly to be locked, and a second spring within the second spring pocket adds tension to the lock plate to further bias the lock plate.

According to further aspects of the present disclosure, a shuttle lock for use with an artificial limb comprises an upper assembly, a lower assembly, and a locking mechanism. The upper assembly includes an aperture for receiving a coupler; a cutout for a channel, wherein the cutout includes a wide portion and a narrow portion; and a groove on a surface of the upper assembly, wherein the groove extends radially from the aperture toward an outer edge of the upper assembly. The lower assembly includes an aperture that aligns with the aperture of the upper assembly when the upper assembly and the lower assembly are coupled together, a platform that surrounds a portion of the aperture of the lower assembly, wherein the platform includes a width, and a first spring pocket located co-radially with the aperture of the lower assembly. A cutout of the lower assembly includes a wide portion and a narrow portion, such that when the upper assembly and the lower assembly are coupled together, the channel is formed. A locking-mechanism pocket extends from the first spring pocket and the second spring pocket and surrounds the aperture of the lower assembly, includes a middle portion, and includes two side portions that flank the middle portion, wherein the middle portion is raised compared to the side portions. A bottom of the lower assembly includes a collar protruding away from a surface of the bottom and surrounding the aperture of the lower assembly and also includes coupling holes. A locking mechanism of the lower assembly includes a lock plate with an aperture that fits around the platform; a pin that fits into the channel and abuts the lock plate such that when a user activates the pin, the lock plate traverses within the locking-mechanism pocket to unlock the shuttle lock; and a first spring within the first spring pocket, wherein the first spring biases the lock plate linearly to be locked.

According to yet further aspects of the present disclosure, a shuttle lock for use with an artificial limb comprises an upper assembly, a lower assembly, and a locking mechanism. The upper assembly includes an aperture for receiving a coupler; a groove on a surface of the upper assembly, wherein the groove extends radially from the aperture toward an outer edge of the upper assembly; and a lip around the outer edge of the upper assembly such that the groove does not extend to the outer edge of the upper assembly. The lower assembly includes an aperture that aligns with the aperture of the upper assembly when the upper assembly and the lower assembly are coupled together, a platform (with a width) that surrounds a portion of the aperture of the lower assembly, a first spring pocket located co-radially with the aperture of the lower assembly, and a channel. The lower assembly further include a locking-mechanism pocket that extends from the first spring pocket and the second spring pocket and surrounds the aperture of the lower assembly, includes a middle portion, and includes two side portions that flank the middle portion, wherein the middle portion is raised compared to the side portions. A bottom of the lower assembly includes a collar that surrounds the aperture of the lower assembly and protrudes away from a surface of the bottom and includes coupling holes. A locking mechanism includes a lock plate with an aperture that fits around the platform; a pin that fits into the channel and abuts the lock plate such that when a user activates the pin, the lock plate traverses within the locking-mechanism pocket to unlock the shuttle lock; and a first spring within the first spring pocket, wherein the first spring biases the lock plate linearly to be locked.

According to still further aspects of the present disclosure, a shuttle lock for use with an artificial limb comprises an upper assembly, a lower assembly, and a locking mechanism. The upper assembly includes an aperture for receiving a coupler, and a groove on a surface of the upper assembly, wherein the groove extends radially from the aperture toward an outer edge of the upper assembly. The lower assembly includes an aperture that aligns with the aperture of the upper assembly when the upper assembly and the assembly portion are coupled together, a platform that surrounds a portion of the aperture of the lower assembly, wherein the platform includes a width, a first spring pocket located co-radially with the aperture of the lower assembly, and a second spring pocket located non-radially with the aperture of the lower assembly, wherein the second spring pocket is longer than the first spring pocket. The lower assembly also includes a channel and a locking-mechanism pocket that extends from the first spring pocket and the second spring pocket and surrounds the aperture of the lower assembly, includes a middle portion, and includes two side portions that flank the middle portion, wherein the middle portion is raised compared to the side portions. A bottom of the lower assembly includes coupling holes and a collar surrounding the aperture of the lower assembly, wherein the collar protrudes away from a surface of the bottom. The locking assembly includes a lock plate with an aperture that fits around the platform and a pin that fits into the channel and abuts the lock plate such that when a user activates the pin, the lock plate traverses within the locking-mechanism pocket to unlock the shuttle lock. The locking mechanism further includes a first spring within the first spring pocket, wherein the first spring biases the lock plate linearly to be locked, and a second spring within the second spring pocket, wherein the second spring adds tension to the lock plate.

According to aspects of the present disclosure, a shuttle lock for use with an artificial limb comprises an upper assembly, a lower assembly, and a locking mechanism. The upper assembly includes an aperture for receiving a coupler, and a groove on a surface of the upper assembly, wherein the groove extends radially from the aperture toward an outer edge of the upper assembly. The lower assembly includes an aperture that aligns with the aperture of the upper assembly when the upper assembly and the lower assembly are coupled together, a platform (with a width) that surrounds a portion of the aperture of the lower assembly, a first spring pocket located co-radially with the aperture of the lower assembly, and a channel. A locking-mechanism pocket of the lower assembly extends from the first spring pocket and the second spring pocket and surrounds the aperture of the lower assembly, includes a middle portion, and includes two side portions that flank the middle portion, wherein the middle portion is raised compared to the side portions and includes a width less than a width of the platform. A bottom of the lower assembly includes coupling holes and a collar surrounding the aperture of the lower assembly, wherein the collar protrudes away from a surface of the bottom. The locking mechanism includes a lock plate with an aperture that fits around the platform; a pin that fits into the channel and abuts the lock plate such that when a user activates the pin, the lock plate traverses within the locking-mechanism pocket to unlock the shuttle lock; and a first spring within the first spring pocket, wherein the first spring biases the lock plate linearly to be locked.

According to aspects of the present disclosure, a shuttle lock for use with an artificial limb comprises an upper assembly, a lower assembly, and a locking mechanism. The upper assembly includes an aperture for receiving a coupler and a groove on a surface of the upper assembly, wherein the groove extends radially from the aperture toward an outer edge of the upper assembly. The lower assembly includes an aperture that aligns with the aperture of the upper assembly when the upper assembly and the lower assembly are coupled together, a platform that surrounds a portion of the aperture of the lower assembly, wherein the platform includes a width, a first spring pocket located co-radially with the aperture of the lower assembly, and a channel. A locking-mechanism pocket of the lower assembly extends from the first spring pocket and the second spring pocket and surrounds the aperture of the lower assembly, includes a middle portion, and includes two side portions that flank the middle portion, wherein the middle portion is raised compared to the side portions. A bottom of the lower assembly includes a collar surrounding the aperture of the lower assembly, wherein the collar protrudes away from a surface of the bottom, coupling holes that are flush with the surface of the bottom, and a sunken portion extending from the collar and terminates before the coupling holes. The locking mechanism includes a lock plate with an aperture that fits around the platform, includes a pin that fits into the channel and abuts the lock plate such that when a user activates the pin, the lock plate traverses within the locking-mechanism pocket to unlock the shuttle lock, and includes a first spring within the first spring pocket, wherein the first spring biases the lock plate linearly to be locked.

### BRIEF DESCRIPTION OF THE SEVERAL VIEWS OF THE DRAWINGS

FIG. 1 is a top view of an upper assembly for a shuttle lock, according to aspects of the present disclosure;

FIG. 2 is a bottom view of the upper assembly for a shuttle lock, according to aspects of the present disclosure;

FIG. 3 is a top view of lower assembly for a shuttle lock, according to aspects of the present disclosure;

FIG. 4 is a bottom view of a lower assembly for a shuttle lock, according to aspects of the present disclosure;

FIG. 5 is a side view of a pin for a shuttle lock, according to aspects of the present disclosure;

FIG. 6 is an exploded view of the shuttle using components from FIGS. 1-5, according to aspects of the present disclosure; and

FIG. 7 is a view of a circular lock plate, according to aspects of the present disclosure.

### DETAILED DESCRIPTION

According to aspects of the present disclosure, a shuttle lock for use with prosthetic limbs includes improvements over existing solutions. The shuttle lock includes an upper assembly that includes a lip around an outer edge of the upper assembly to stop a coupler from an artificial limb from sliding off the upper assembly, if the coupler is sliding away from a center of the upper assembly when attaching the artificial limb to the shuttle lock.

The shuttle lock also includes a lower assembly that includes two spring pockets: a first spring pocket that is aligned radially with an aperture of the lower assembly, and a second spring pocket that is substantially parallel to the first spring pocket, not radially aligned with the aperture, and larger than the first spring pocket. Springs with the same length and spring constant can be used in both spring pockets to supply a bias (or tension) to a lock plate. Another improvement on the lower assembly includes a locking mechanism pocket (where the lock plate resides) with a narrower raised portion than existing solutions, which balances a desire for a lock plate that is not required to be perfectly flat (as a lock plate may bend after insertion in the shuttle lock) while also minimizing rocking of the lock plate during use.

Moreover, a bottom of the lower portion includes coupling holes that are flush with a surface of the bottom, which differs from existing solutions that include collars around the coupling holes. The bottom also includes a sunken portion extending from the center of the lower assembly but terminating before the coupling holes, which helps when coupling the lower assembly to an uneven surface.

Further, a pin for the shuttle lock includes a wide portion and a narrow portion, where the wide portion can be rotated to set the pin to a locked state or released state with respect to the lock plate. The wider portion is cylindrical, does not include pins at an end, and has a portion housed in the upper and lower assemblies, which reduces a likelihood that the pin would snag on anything.

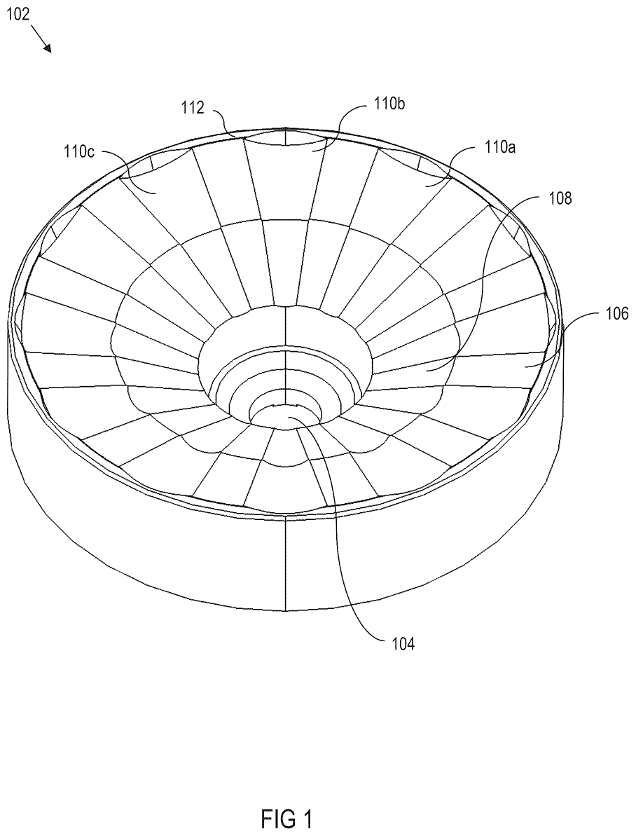

Turning now to the figures, and in particular FIG. 1, an upper assembly 102 for a shuttle lock is shown. The upper assembly 102 is round and includes an aperture 104 for receiving a coupler of a limb prosthetic implant to be locked with the shuttle lock. A surface of the upper assembly slopes toward the aperture and may include two different angles of slope, where a first slope 106 is at a first angle toward the aperture 104, and a second slope 108 is at a second angle toward the aperture 104. While two slopes are shown in FIG. 1, any number of slopes may be used—even just one.

Further, the surface includes several grooves 110a, 110b, 110c (collectively 110, and not all grooves are labelled so FIG. 1 is less cluttered) that extend radially from the aperture 104. The grooves 110 help guide a coupler of a prosthetic limb to the aperture 104, so the prosthetic limb may be secured with the shuttle lock, as described herein. Moreover, a lip 112 extends around an outer edge of the upper assembly 102, which prevents the coupler of the prosthetic limb from sliding off the surface (e.g., out of the groove 110) if the coupler of the prosthetic limb slides away from the aperture 104.

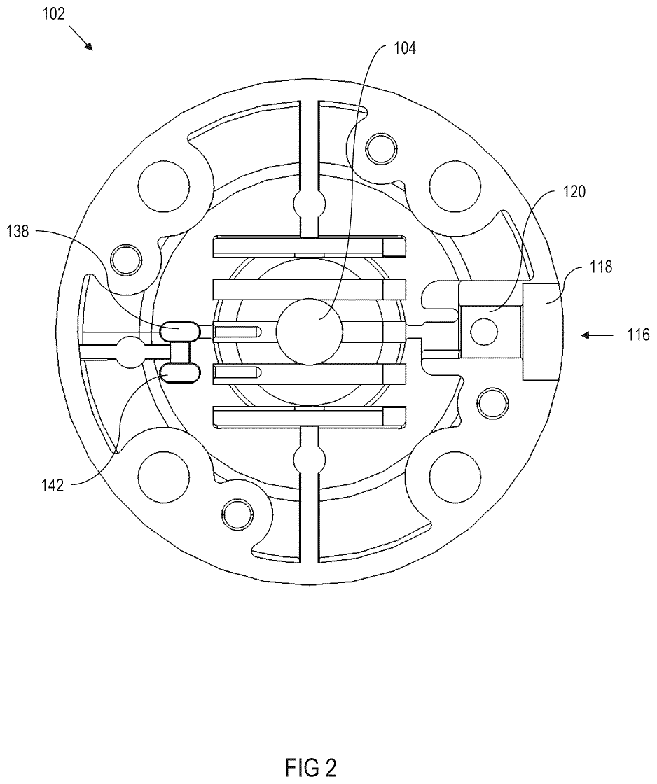

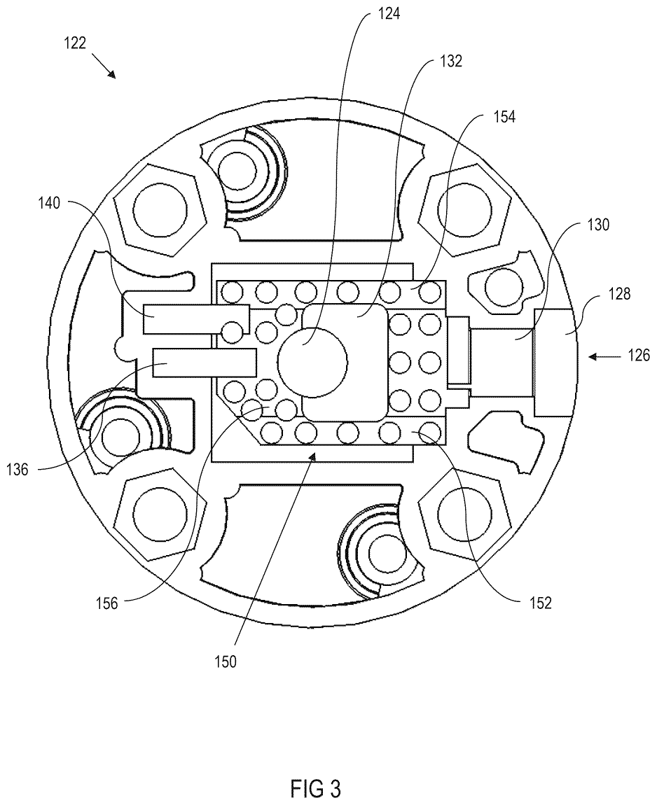

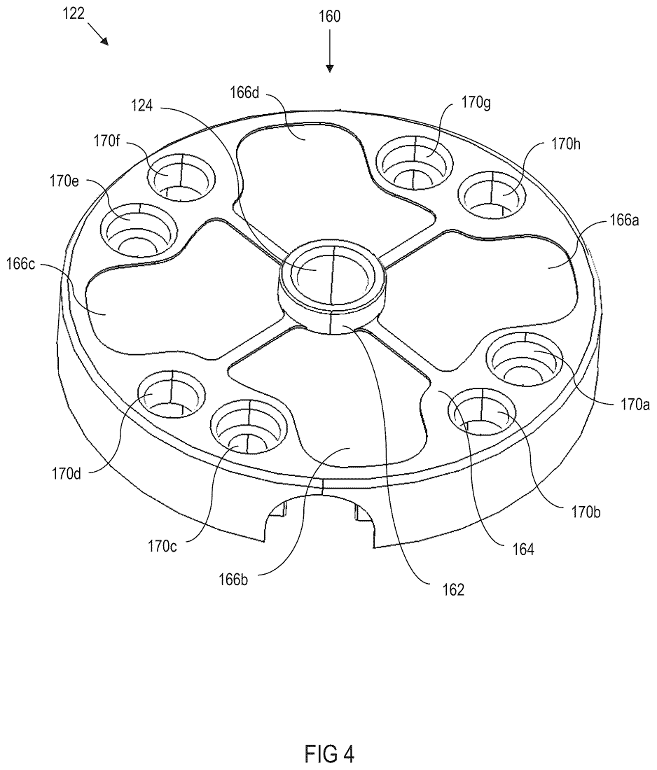

Turning now to FIG. 2, a bottom of the upper assembly 102 is shown. The bottom of the upper assembly 102 includes a cutout 116 for a channel that is created when the upper assembly 102 is coupled to a lower assembly (see FIGS. 3-4). The cutout 116 includes a wide portion 118 and a narrow portion 120.

Turning also to FIG. 3, a lower assembly 122 is shown. The lower assembly 122 includes an aperture 124 that aligns with the aperture 104 of the upper assembly 102 when the upper assembly 102 and the lower assembly 122 are coupled together. Similar to the upper assembly 102, the lower assembly 122 also includes a cutout 126 for the channel that is created when the upper assembly 102 is coupled to the lower assembly 122. The cutout 126 includes a wide portion 128 and a narrow portion 130. As the channel takes the shape of the cutouts, the channel has a wide portion and a narrow portion. The narrow portion may be cylindrical, and the wide portion may be cylindrical, polygonal, etc.

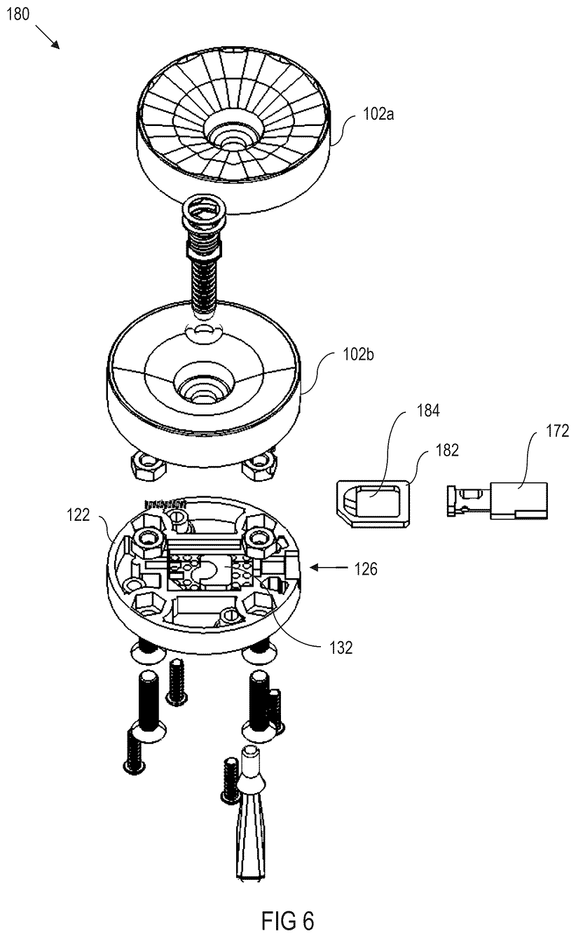

Further, the lower assembly 122 includes a platform 132 that surrounds a portion of the aperture 124. The platform 132 includes a width that spans the entire aperture 124 and guides a lock plate (discussed in reference to FIG. 6, below) to ensure that the lock plate traverses linearly when activated.

A first spring pocket 136 of the lower assembly 122 is located co-radially with the aperture 124, and the upper assembly 102 includes a cap 138 that covers a portion of the first spring pocket 136. Also, a second spring pocket 140 of the lower assembly 122 is located non-radially with the aperture 124, and the upper assembly 102 includes a cap 142 that covers a portion of the second spring pocket 140. The first and second spring pockets 136, 140 are opposite the cutout 126 (i.e., on the other side of the aperture 124) and are different sizes (e.g., the second spring pocket 140 is longer than the first spring pocket 136), which allows for springs of the same size and same spring constant to be used in those spring pockets 136, 140 to provide different force on the lock plate when the lock plate is in the lower assembly 122. For example, if the second spring pocket 140 is longer than the first spring pocket 136 and identical springs are placed in both spring pockets 136, 140, then the spring in the first spring pocket 136 will have more influence (i.e., more force applied) on the lock plate than the spring in the second spring pocket 140.

Moreover, the lower assembly 122 includes a locking-mechanism pocket 150 that extends from the spring pockets 136, 140 and surrounds the aperture 124 of the lower assembly 122. The locking-mechanism pocket 150 includes two side portions 152, 154 and a middle portion 156, where the middle portion 156 is slightly raised (e.g., 3/5000 inch or 0.015 mm) above the two side portions 152, 154. Further, the middle portion 156 includes a width that is less than the width of the platform 132. In other words, when looking down from the top, the platform 132 extends out over the two side portions 142, 154. When the lock plate (see FIG. 6) is placed in the locking-mechanism pocket 150, the lock plate rests on the middle portion 156 and will extend out over the side portions 152, 154. The side portions 152, 154 being slightly lower than the middle portion 156 allow for the lock plate not to bend once the lock plate is inserted in the locking-mechanism pocket 150. As shown in FIG. 3, the locking-mechanism pocket 150 is a rectangle with a cut corner to allow a corresponding keyed lock plate (see 182, FIG. 6 below) to be fit into the locking-mechanism pocket 150 in only one direction. However, other shapes are possible as an oblong shape with flat length sides (to allow the lock plate to slide) with a keyed corner which could house a mostly circular lock plate with a keyed (e.g., flattened) portion.

Turning to FIG. 4, a bottom 160 of the lower assembly 122 is shown. The bottom 160 includes a collar 162 that surrounds the aperture 124 protrudes away from a surface 162 of the bottom 160. The bottom 160 further includes sunken portions 166a-d (collectively 166) that extend from the collar 162. Moreover, the bottom 160 includes coupling holes 170a-h (collectively 170) that are used to couple the lower assembly to the upper assembly. These coupling holes 170 are flush with the surface 164 (i.e., all the coupling holes 170 do not include a collar), and the sunken portions 166 terminate before they reach the coupling holes 170. The sunken portions 166 help when attaching the shuttle lock to an uneven surface without relying on collars around the coupling holes.

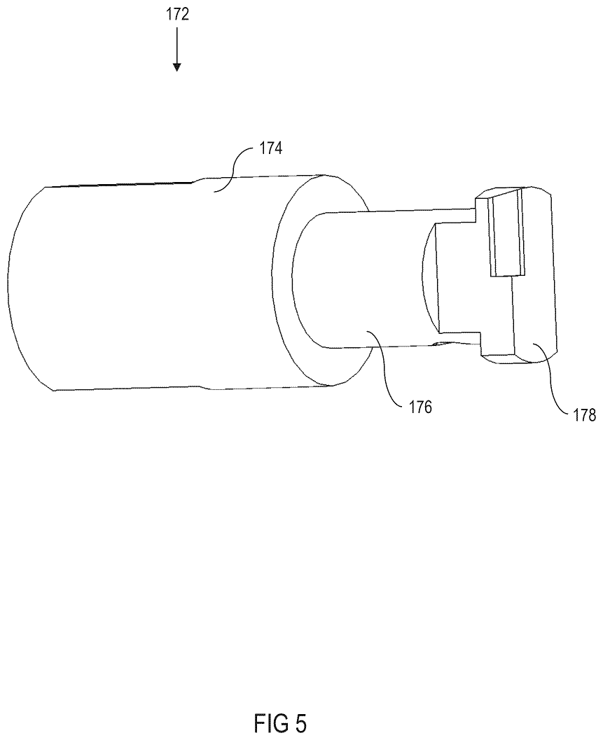

Turning now to FIG. 5, a pin 172 for a locking mechanism of the shuttle lock is shown. The pin 172 includes a wide portion 174, a narrow portion 176, and a locking portion 178. The pin 172 fits into the channel created by the cutouts (116, FIG. 2 and 126, FIG. 3) when the upper assembly and the lower assembly are coupled together. While the pin 172 is in the channel, a portion of the pin that extends outside the channel does not include any projection portions that may get snagged on an object outside the shuttle lock. When a user pushes in on the pin 172, the lock plate (see FIG. 6) traverses within the locking-mechanism pocket (150, FIG. 3) of the lower assembly. When a user rotates the pin 172, the locking portion 178 secures the pin 172.

Turning now to FIG. 6, an exploded view of the shuttle lock 180 is shown. In FIG. 6 there are two options for an upper assembly 102: an upper assembly 102a with grooves, or an upper assembly 102b without grooves. Only one upper assembly 102 will be used at any given time on the shuttle lock 180. The lower assembly 122 includes the platform 132 and may include the other elements discussed above in reference to FIGS. 3-4. Further, the pin 172 is inserted into the cutout 126 of the lower assembly 122 or the cutout (116, FIG. 2) of the upper assembly 102 before coupling the upper assembly 102 to the lower assembly 122. Moreover, a lock plate 182 sized to fit into the locking-mechanism pocket (150, FIG. 3) with an aperture 184 sized to fit around the platform 132 while allowing for linear movement when pushed by the pin 172. Further, the locking plate 182 is biased by the springs of the spring pockets (136, 140, FIG. 3).

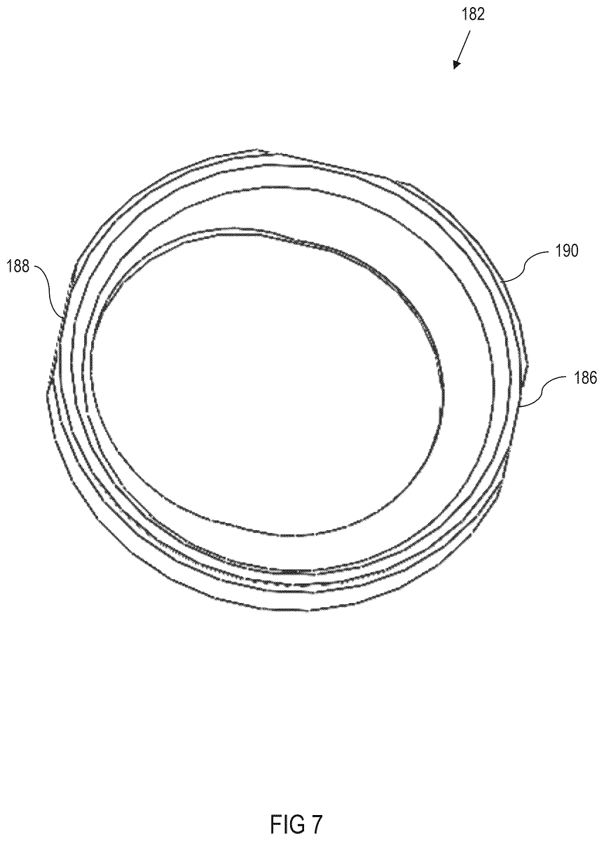

As shown in FIG. 6, the lock plate 182 is generally rectangular with a keyed corner that corresponds to a keying in the locking-mechanism pocket (see 150, FIG. 3). However, the lock plate may be any desired shape that corresponds to whatever shape the locking-mechanism pocket may be. For example, in FIG. 7, the lock plate 182 is a generally circular shape with a flat portions 186, 188 as a key. Further, one or more edges 190 of the lock plate 182 may be raised more than a rest of the lock plate to provide more stability and less rocking in the pocket when in use. The circular lock plate 182 of FIG. 7 functions similarly to the lock plate 182 of FIG. 6.

The improvements discussed herein may be used in a shuttle lock individually or in combination with one or more others of the improvements discussed herein.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. Aspects of the disclosure were chosen and described in order to best explain the principles of the disclosure and the practical application, and to enable others of ordinary skill in the art to understand the disclosure for various embodiments with various modifications as are suited to the particular use contemplated.

What is claimed is:

1. A shuttle lock for use with an artificial limb, the shuttle lock comprising:
an upper assembly including:
an aperture for receiving a coupler;
a cutout for a channel, wherein the cutout includes a wide portion and a narrow portion;
a groove on a surface of the upper assembly, wherein the groove extends radially from the aperture toward an outer edge of the upper assembly; and
a lip around the outer edge of the upper assembly such that the groove does not extend to the outer edge of the upper assembly;
a lower assembly including:
an aperture that aligns with the aperture of the upper assembly when the upper assembly and the lower assembly are coupled together;
a platform that surrounds a portion of the aperture of the lower assembly, wherein the platform includes a width;
a first spring pocket located co-radially with the aperture of the lower assembly;
a second spring pocket located non-radially with the aperture of the lower assembly, wherein the second spring pocket is longer than the first spring pocket;
a cutout, wherein the cutout includes a wide portion and a narrow portion, such that when the upper assembly and the lower assembly are coupled together, the channel is formed;
a locking-mechanism pocket that:
extends from the first spring pocket and the second spring pocket and surrounds the aperture of the lower assembly;
includes a middle portion; and
includes two side portions that flank the middle portion, wherein the middle portion is raised compared to the side portions and includes a width less than a width of the platform;
a bottom including:
a collar surrounding the aperture of the lower assembly, wherein the collar protrudes away from a surface of the bottom;
coupling holes that are flush with the surface of the bottom; and
a sunken portion extending from the collar and terminates before the coupling holes; and
a locking mechanism including:
a lock plate with an aperture that fits around the platform;
a pin that fits into the channel and abuts the lock plate such that when a user activates the pin, the lock plate traverses within the locking-mechanism pocket to unlock the shuttle lock;
a first spring within the first spring pocket, wherein the first spring biases the lock plate linearly to be locked; and
a second spring within the second spring pocket, wherein the second spring adds tension to the lock plate.

2. The shuttle lock of claim 1, wherein the narrow portion of the channel is cylindrical.

3. The shuttle lock of claim 2, wherein the wide portion of the channel is cylindrical.

4. The shuttle lock of claim 2, wherein the wide portion of the channel is polygonal.

5. The shuttle lock of claim 1, wherein the first spring has a same length and spring constant as the second spring.

6. A shuttle lock for use with an artificial limb, the shuttle lock comprising:
an upper assembly including:
an aperture for receiving a coupler; and
a groove on a surface of the upper assembly, wherein the groove extends radially from the aperture toward an outer edge of the upper assembly;
a lower assembly including:
an aperture that aligns with the aperture of the upper assembly when the upper assembly and the lower assembly are coupled together;
a platform that surrounds a portion of the aperture of the lower assembly, wherein the platform includes a width;
a first spring pocket located co-radially with the aperture of the lower assembly;
a channel;
a locking-mechanism pocket that:
extends from the first spring pocket and the second spring pocket and surrounds the aperture of the lower assembly;
includes a middle portion; and
includes two side portions that flank the middle portion, wherein the middle portion is raised compared to the side portions and includes a width less than a width of the platform;
a bottom including:
a collar surrounding the aperture of the lower assembly, wherein the collar protrudes away from a surface of the bottom; and
coupling holes; and
a locking mechanism including:
a lock plate with an aperture that fits around the platform;
a pin that fits into the channel and abuts the lock plate such that when a user activates the pin, the lock plate traverses within the locking-mechanism pocket to unlock the shuttle lock; and
a first spring within the first spring pocket, wherein the first spring biases the lock plate linearly to be locked.

7. The shuttle lock for use with an artificial limb wherein:
the upper assembly further includes:
a cutout for a channel, wherein the cutout includes a wide portion and a narrow portion; and
the lower assembly further includes:
a cutout, wherein the cutout includes a wide portion and a narrow portion, such that when the upper assembly and the lower assembly are coupled together, the channel is formed.

8. The shuttle lock for use with an artificial limb of claim 6, wherein::
the upper assembly further includes:
a lip around the outer edge of the upper assembly such that the groove does not extend to the outer edge of the upper assembly.

9. The shuttle lock for use with an artificial limb of claim 6, wherein:
the lower assembly further includes:
a second spring pocket located non-radially with the aperture of the lower assembly, wherein the second spring pocket is longer than the first spring pocket; and
the locking mechanism further includes:
a second spring within the second spring pocket, wherein the second spring adds tension to the lock plate.

10. The shuttle lock for use with an artificial limb of claim 6, wherein:
the bottom of the lower assembly further includes:
a sunken portion extending from the collar and terminates before the coupling holes.
